(19) United States
(12) Patent Application Publication
(10) Pub. No.: US 2009/0105581 A1
(43) Pub. Date: Apr. 23, 2009

(54) ULTRASOUND IN MAGNETIC SPATIAL IMAGING APPARATUS

(76) Inventor: Gerold Widenhorn, Uberlingen (DE)

Correspondence Address: SUGHRUE MION, PLLC, 2100 PENNSYLVANIA AVENUE, N.W., SUITE 800, WASHINGTON, DC, 20037, US

(21) Appl. No.: 12/282,854

(22) PCT Filed: Mar. 1, 2007
(86) PCT No.: PCT/AU07/00233

§ 371 Date: Sep. 12, 2008

(30) Foreign Application Priority Data
Mar. 15, 2006 (AU) 2006901321

(51) Int. Cl. A61B 5/055; A61B 8/00

(52) U.S. Cl. 600/411; 600/437

(57) ABSTRACT

The present invention provides apparatus and methods for acquiring ultrasound measurements representing biosignals from a subject located in the strong magnetic field of a spatial imaging device. The invention includes ultrasound probes having minimal magnetic parts and an ultrasound signal pre-amplifier being shielded by a barrier or barriers from the strong magnetic field of a spatial imaging device such as an MRI, PET, or CT scanner. Most preferably, the ultrasound probe produces ultrasound waves in the frequency range of approximately 2.0 MHz to 2.5 MHz. The ultrasound probe may include no electronic components and contains minimal magnetic or ferromagnetic parts. The elements for acquiring and analysing ultrasound waves may be located in separate rooms from the imaging device. The invention provides a method of taking ultrasound measurements in quick succession with measurements of a spatial imaging device to provide a more informative understanding of the physiology of the subject.

### FIELD OF THE INVENTION

This invention relates to methods and apparatus for measuring physiological parameters, in particular blood flow rates and electrophysiological activity, using ultrasound and spatial imaging techniques.

### BACKGROUND

Developments in imaging techniques have made it possible to measure physiological parameters over differing timeframes by exploiting the physics of waveforms as they pass through heterogeneous tissues. For example, it is known in the art that the properties of the diffraction of sound waves of very high frequency (known as the Doppler effect) directed toward moving fluids, including blood moving through vessels, can be analysed to measure blood flow-rate. Such techniques using sound waves (known as ultrasound technologies) have been conveniently used in applications of cardiology and, more recently, brain blood-flow. Further developments in the use of ultrasound in brain blood-flow measurements are known in the art where measurements are made using ultrasound penetrating the cranium (Transcranial Doppler, or TCD). Such techniques were disclosed by Aaslid in U.S. Pat. No. 4,817,621, which described apparatus for TCD to measure blood-flow in vessels in the brain.

Other developments for making images of tissue in situ have occurred independently, using different principles of physics, such as measuring concentrations of particular atoms and molecules with varying concentrations in heterogeneous tissues by activating the atoms and molecules by strong-magnetic fields and measuring the activation. Such a technique is known in the art of magnetic resonance imaging (MRI).

Both ultrasound and MRI are used to measure aspects of fluids in tissues. However, the techniques, like other imaging techniques, operate on different timescales and can be interpreted for different physiological parameters. The speed with which ultrasound waves can be generated, reflected and analysed is over periods of milliseconds. The speed at which MRI images can be generated is at least tenfold slower. Ultrasound measurements show immediate and dynamically changing flow rates of fluids such as blood. Ultrasound measurements inform little about anatomy or morphology of the tissues being penetrated. MRI measurements are static but very rich in anatomical and morphological information.

A great advantage would occur if physiological information could be derived from both ultrasound and MRI measurements of a tissue. This would be particularly advantageous for TCD blood-flow measurements of brain tissue made concurrently with MRI structural measurements. A device and method for concurrently taking ultrasound and MRI measurements, or at least in quick succession, of the same tissue could collectively provide much more information that independent, non-concurrent measurements made using either technique.

While making concurrent or quick successive measurements of physiological parameters using ultrasound and other imaging techniques like MRI would be advantageous, it has not been possible to date. A major reason is because the strong magnetic fields of MRI induce currents in electrical conducting materials. Such conducting materials are commonly used in ultrasound devices. The currents interfere with, and make it impossible to generate and receive, ultrasound waves for analysis. What is needed is ultrasound apparatus, In particular TCD apparatus, that is unaffected, or minimally affected by the magnetic fields of imaging systems such as MRI systems, to enable the concurrent or quick successive measurements of physiological parameters to enable independent and dynamic measurements of physiological processes. Similarly, methods for concurrently or quick successive measuring physiological parameters with ultrasound and MRI are needed. Such methods and apparatus would have many applications such as measuring blood flow during disease events such as stroke which is associated with abnormal blood flows in the brain.

### SUMMARY OF THE INVENTION

It is known in the art that ultrasound transducer probes for generating, transmitting and receiving ultrasound waves are comprised of magnetic materials including a coil for amplifying the reflected ultrasound signal received by the probe. Conventionally within the member is a crystal that vibrates to produce the ultrasound waves, the crystal being adjacent or nearly adjacent the amplifying coil needed to amplify signals for processing. However, placing such a probe within the magnetic field of magnetic imaging system such as an MRI scanner results in the induction of electrical currents in the coils by the magnetic field of the imaging system, the result being that the ultrasound probe is unusable for generating and receiving meaningful ultrasound signals. Surprisingly, the invention provides that the received ultrasound signal amplifier can be spatially separated from the ultrasound-generating crystal, outside the strong magnetic field of the imaging system, but in electrical communication with a suitable conducting connector so that the received ultrasound signal may be amplified and further processed outside the magnetic field. The result is that ultrasound signals representative of physiological processes can be measured in quick succession with the operation of the magnetic imaging system, notwithstanding the presence of the ultrasound member within the magnetic field of the magnetic imaging system.

The invention most advantageously provides apparatus and methods for measuring both ultrasound signals and magnetic imaging modalities. In one aspect the invention most advantageously provides a method of making at least one ultrasound imaging measurement using ultrasound apparatus within the Magnetic field of a spatial imaging device having a magnetic field successively with making magnetic spatial images with the spatial imaging device, the method including the steps of:
a. positioning at least one non-magnetic ultrasound member adjacent a subject for making an ultrasound measurement;
b. establishing electrical communication with the at least one non-magnetic ultrasound member with an ultrasound signal pre-amplifier;
c. magnetically Isolating the magnetic field of the spatial-image device from said signal pre-amplifying means;
d. operating the at least one ultrasound member and an ultrasound imaging means to create a physiological signal of fluid flow in the subject;
e. interrupting the operation of the ultrasound member and ultrasound imaging means;
f. operating the spatial imaging device to record an image during said interruption; and
g. repeating steps e and f, if desired.

In another aspect, the invention provides apparatus for making ultrasound measurements of fluid flow in a subject located in a spatial imaging device having a strong magnetic field, including: a least one ultrasound member for generating ultrasound waves, said member having minimal magnetic parts; electrical signal communication means in communication with the at least one ultrasound member; electrical signal amplifying means; magnetic field barrier for magnetically shielding the signal amplifying means; and an imaging device having a strong magnetic field wherein said magnetic field barrier is disposed to shield said signal amplifying means from said magnetic file of said imaging device. Preferably wherein the ultrasound member is an ultrasound probe for producing ultrasound waves in the frequency range of approximately 1 MHz to 4 MHz. More preferably, the ultrasound probe for produce ultrasound waves in the frequency range of approximately 2.0 MHz to 2.5 MHz. Preferably, the ultrasound member includes no electronic components. Preferably, the apparatus contains minimal magnetic or ferromagnetic parts in the ultrasound member. The ultrasound member may include conducting parts comprised of carbon materials. Other embodiments include parts in the ultrasound member being comprised of substances that minimise artefacts or distortion to the image modality image and analysis processing. The elements for acquiring and analysing ultrasound waves may be located in separate rooms from the imaging device wherein the magnetic field barrier is the wall of an enclosure. Preferably, the apparatus includes a headband for positioning at least one ultrasound member for taking ultrasound measurements. Preferably, the headband is comprised of a nonmagnetic material or materials. Preferably the spatial imaging device is any one of an MRI scanner, PET scanner, or CT scanner.

### BRIEF DESCRIPTION OF THE FIGURES

FIG. 1 shows a diagram of the features of the invention.

FIG. 2 shows an embodiment of non-magnetic ultrasound members for use in magnetic fields.

FIG. 3 shows a side view of a non-magnetic ultrasound member held in place with a non-magnetic band for use in magnetic fields.

FIG. 4 shows a front view of two non-magnetic ultrasound members held in place with a non-magnetic band for use in magnetic fields.

### DETAILED DESCRIPTION OF THE FIGURES AND MOST PREFERRED EMBODIMENTS

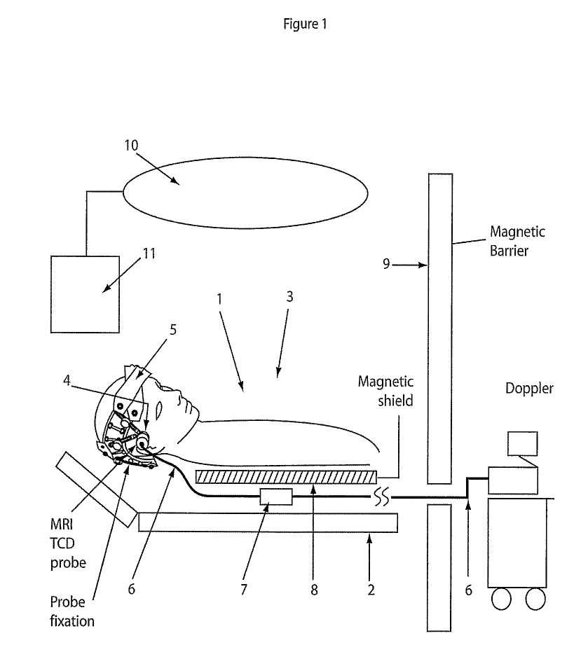

The invention is most easily understood with reference to the accompanying figures. FIG. 1 shows an embodiment of the invention, including the elements necessary to acquire ultrasound signals and spatial imaging information using strong magnetic fields. It will be understood that other embodiments of the invention are possible and that the scope of the invention is not limited to the embodiments described herein. In FIG. 1 is shown a subject 1 in a prone position on a table 2 within the magnetic field 3 of a spatial imaging device 10. The spatial imaging device may be any suitable spatial imaging device having a magnetic field. Preferably, the spatial imaging device is an MRI device. Other spatial imaging devices such as PET or CT devices are suitable for practising the invention. An ultrasound member 4 is engaged with a band 5 which, in turn, is engaged with the head 5 of the subject 1. Only one ultrasound member is shown in FIG. 1 but it is possible that more than one member may be used in practising the invention. In electrical communication with the ultrasound member 4 is a conducting lead 6 which communicates signals to a preamplifier 7 that is located on the side of a magnetic field barrier or shield 8 opposite to the magnetic field 3. The magnetic field barrier 8 operates to shield the preamplifier 7 from electrical interference caused by induction of current in the pre-amplifier and conducting lead 6. The conducting lead 6 transverses a second magnetic field barrier 9, which shields the ultrasound-signal analysis device from interference caused by the strong magnetic field 3. It is possible to practise the invention without a second magnetic field barrier. Preferably there are two magnetic field barriers as shown in FIG. 1. Preferably, the ultrasound-signal analysis device is a Doppler-Box. The arrangement of the elements herein described allows an ultrasound measurement to be made with the co-operation of the ultrasound elements within and without the magnetic field 3 at a point in time. At alternate points in time, the operation of the ultrasound equipment is paused and spatial images of the subject may be made with the spatial imaging device.

The invention is best performed when the distance between the transducer crystal of the ultrasound member 4 and the pre-amplifier is as short as possible to ensure that adequately measurable ultrasound signals can be acquired. This is achieved by placing the preamplifier 7 at the shielded area on the shielded side of the magnetic field shield 7 close to the tissue of interest with a connector for the member. This is best achieved with ultrasound members 4 having long conducting leads B.

The apparatus of the invention includes ultrasound members that may be spatially separate from the ultrasound signal processing and analysing apparatus, preferably including being located in separate rooms to minimise interference from the magnetic field of the magnetic imaging system, wherein the magnetic field shield is a wall of an enclosure such as a room. This is shown in FIG. 1 with the magnetic shield 9. In one embodiment of the invention, using an ultrasound Doppler-Box, it is also possible to locate the ultrasound Doppler-Box close to the spatial imaging device.

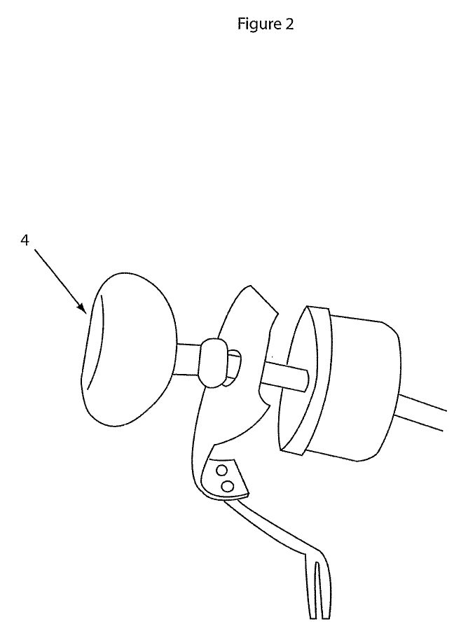

Any suitable ultrasound member may be used. Preferably the ultrasound members are probes which produce ultrasound waves within the frequency range of 1 MHz to 3 MHz. More preferably the ultrasound members are 2 MHz to 2.5 MHz ultrasound probes. FIG. 2 shows an embodiment of a suitable ultrasound member 4. Preferably, the member 4 contains no electronic elements within.

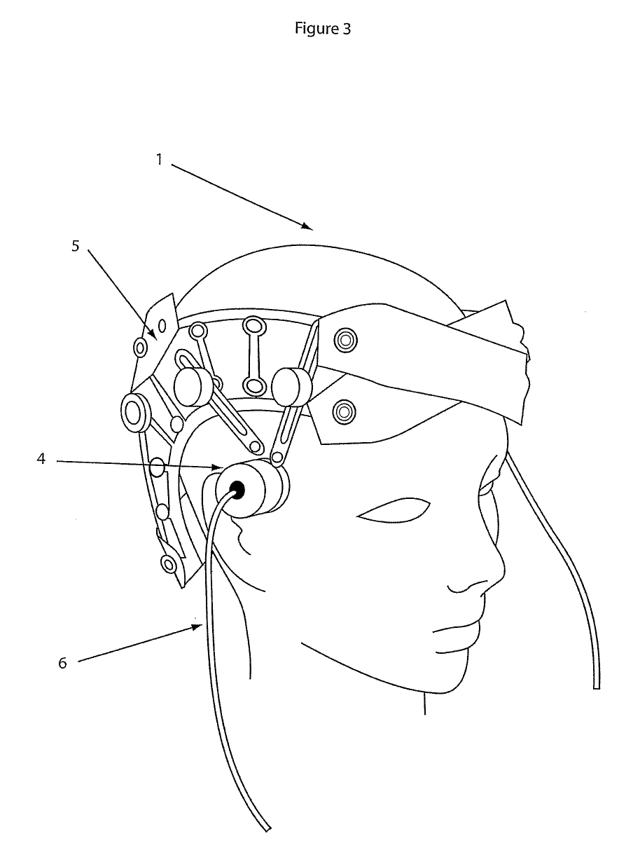

FIG. 3 shows a side view of the head of a subject 1 with a headband 5 for ultrasound sonication engaged with an ultrasound member 4 in fixed in position for sonication. Preferably, all parts of the headband 5 are constructed of non-magnetic materials.

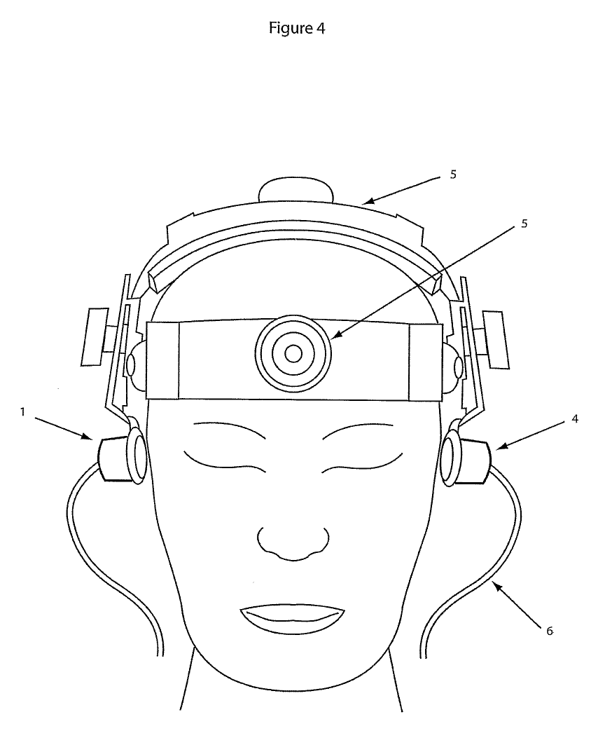

FIG. 4 shows a front view of the head of a subject 1 with an alternative embodiment of the invention with an ultrasound member 4 on each side of the head of the subject. A conducting lead 6 in conducting communication with the ultrasound member carries signals from each of the ultrasound members.

It will be understood that the invention is not limited to combining ultrasound apparatus with MRI apparatus but also includes apparatus for other spatial imaging devices including strong magnetic fields such as those implementing CT and PET.

1. A method of making ultrasound imaging measurement using an ultrasound apparatus within the magnetic field of a spatial imaging device having a magnetic field successively with making magnetic spatial images with the spatial imaging device, the method including the steps of:
a. positioning at least one non-magnetic ultrasound member adjacent a subject for making an ultrasound measurement;
b. establishing electrical communication with the at least one non-magnetic ultrasound member with an ultrasound signal pre-amplifier;
c. magnetically isolating the magnetic field of the spatial-image device from said signal pre-amplifying means:
d. operating the at least one ultrasound member and an ultrasound imaging means to create a physiological signal of fluid flow in the subject;
e. interrupting the operation of the ultrasound member and ultrasound imaging means: and
f. operating the spatial imaging device to record an image during said interruption.

2. Apparatus for making ultrasound measurements of fluid flow in a subject located in an imaging device having a strong magnetic field, including:
a. a least one ultrasound member for generating ultrasound waves, said member having minimal conductive parts;
b. electrical signal communication means in communication with the at least one ultrasound member;
c. electrical signal amplifying means;
d. magnetic field barrier for magnetically shielding the signal amplifying means; and
e. an imaging device having a strong magnetic field wherein said magnetic field barrier is disposed to shield said signal amplifying means from said magnetic file of said imaging device.

3. The apparatus of claim 2 wherein said ultrasound member is an ultrasound probe for producing ultrasound waves in the frequency range of approximately 1 MHz to 4 MHz.

4. The apparatus of claim 2 wherein said ultrasound member is an ultrasound probe for producing ultrasound waves in the frequency range of approximately 2.0 MHz to 2.5 MHz.

5. The apparatus of claim 2 wherein the ultrasound member includes no electronic components.

6. The apparatus of claim 2, wherein the magnetic field barrier is a wall of an enclosure.

7. The apparatus of claim 2, further comprising a Doppler box.

8. The apparatus of claim 2, further comprising a headband far positioning at least one ultrasound member for taking ultrasound measurements.

9. The apparatus of claim 8 wherein said headband is comprised of a non-magnetic material or materials.

10. The apparatus of claim 2, wherein the conducting parts of the ultrasound member are comprised of carbon materials.

11. The apparatus of claim 2, wherein said imaging device is any one of an MRI scanner, PET scanner, or CT scanner.

12. The apparatus of claim 2 wherein said ultrasound member is an ultrasound probe for producing ultrasound waves in the frequency range of approximately 1 MHz to 4 MHz.

13. The apparatus of claim 2 wherein said ultrasound member is an ultrasound probe for producing ultrasound waves in the frequency range of approximately 2.0 MHz to 2.5 MHz.

14. The apparatus of claim 2, wherein the ultrasound member includes no electronic components.

15. The apparatus of claim 2, wherein the magnetic field barrier is a wall of an enclosure.

16. The apparatus of claim 2, further comprising a Doppler box.

17. The apparatus of claim 2, further comprising a headband far positioning at least one ultrasound member for taking ultrasound measurements.

18. The apparatus of claim 8, wherein said headband is comprised of a non-magnetic material or materials.

19. The apparatus of claim 2, wherein the conducting parts of the ultrasound member are comprised of carbon materials.

20. The apparatus of claim 2, wherein said imaging devices is any one of an MRI scanner, PET scanner, or CT scanner.
